(19) United States
(12) Patent Application Publication
(10) Pub. No.: US 2024/0065843 A1
(43) Pub. Date: Feb. 29, 2024

(54) Magnesium-Based Porous Coating for Orthopedic Implant

(71) Applicant: Bone Solutions, Inc., Colleyville, TX (US)

(72) Inventor: Frankie Morris, Colleyville, TX (US)

(21) Appl. No.: 18/261,133

(22) PCT Filed: Jan. 24, 2022
(86) PCT No.: PCT/US2022/013469

§ 371 Date: Jul. 12, 2023

Related U.S. Application Data
(60) Provisional application No. 63/145,156, filed on Feb. 3, 2021.

(51) Int. Cl. A61F 2/28; A61L 27/54; A61L 27/56; A61L 31/08

(52) U.S. Cl. CPC A61F 2/2846; A61L 27/54; A61L 27/56; A61L 31/086; A61L 2420/04

(57) ABSTRACT

The present disclosure relates to an orthopedic implant. The orthopedic implant includes an elongated member having a first end and a second end opposite the first end. The orthopedic implant also includes a porous coating secured to an exterior surface of the elongated member. The porous coating includes magnesium phosphate.

### RELATED APPLICATIONS

This application claims priority to U.S. Provisional Application No. 63/145,156 entitled “Magnesium-Based Porous Coating for Orthopedic Implant,” filed on Feb. 3, 2021, the contents of which are hereby incorporated by reference in its entirety.

### TECHNICAL FIELD

The present disclosure relates to an orthopedic implant and methods of use. Embodiments of the orthopedic implant are coated with a magnesium-based porous coating, and can be used for fixing various devices to or in a bone of a patient.

### BACKGROUND

Unless otherwise indicated herein, the materials described in this section are not admitted to be prior art to the claims in this application.

Many procedures in the field of orthopedics require the use of screws, anchors, pins, or other such fixation devices. In one example, such fixation devices may be used to attach soft tissue such as ligaments, tendons, or muscles to a surface from which the soft tissue has become detached. For example, the rotator cuff may be reattached to the humeral head during a shoulder repair. As another example, fixation devices may be used in the reconstruction of the anterior cruciate ligament (ACL) to secure a substitute ligament to the tibia and the femur. Fixation devices may also be used to secure soft tissue to supplementary attachment sites for reinforcement. For example, in urological applications, fixation devices may be used in bladder neck suspension procedures to attach a portion of the bladder to an adjacent bone surface. Such soft tissue attachments may be done during either open or closed surgical procedures, the latter being generally referred to as arthroscopic or endoscopic surgery. The terms “arthroscopic” and “endoscopic” may be used interchangeably herein and are intended to encompass arthroscopic, endoscopic, laparoscopic, hysteroscopic or any other similar surgical procedures performed with elongated instruments inserted through small openings in the body. Many other potential uses of various fixation devices are possible as well.

In some instances, orthopedic implants may be positioned within a cavity formed in a bone of a patient. A tight fit is desired between the orthopedic implant and the surrounding bone forming the cavity wall in order to provide maximum fixation in the shortest time, by maximizing implant stability and the opportunity for bone ingrowth. If there is a gap between the orthopedic implant and the surrounding bone forming the cavity wall, certain problems may occur. For successful orthopedic implants, sufficiently regenerated bone fills the gap between the orthopedic implant and the host bone, so that the orthopedic implant is firmly attached to the surrounding bone. To overcome problems with implant loosening, orthopedic implants need to stimulate rapid bone regeneration in order to replenish the missing bone and/or to fix the orthopedic implant firmly within the host bone.

### SUMMARY

In view of the foregoing, the inventors recognized that an improved orthopedic implant with a magnesium-based porous coating would be desirable. The present invention provides such a device and method of use.

In a first aspect, the present disclosure provides an orthopedic implant. The orthopedic implant includes an elongated member having a first end and a second end opposite the first end. The orthopedic implant also includes a porous coating secured to an exterior surface of the elongated member. The porous coating includes magnesium phosphate.

In a second aspect, the present invention provides a method for securing an orthopedic implant to a bone, the method comprising: (a) providing the orthopedic implant of the first aspect, (b) forming a cavity in the bone, and (c) inserting the second end of the elongated member of the orthopedic implant into the cavity in the bone.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a top view of an orthopedic implant, according to an exemplary embodiment.

FIG. 2 illustrates a side view of the orthopedic implant of FIG. 1, according to an exemplary embodiment.

FIG. 3 illustrates a perspective view of the orthopedic implant of FIG. 1, according to an exemplary embodiment.

FIG. 4 illustrates a perspective view of another orthopedic implant, according to an exemplary embodiment.

FIG. 5 illustrates a perspective view of another orthopedic implant, according to an exemplary embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The exemplary embodiments described herein are not meant to be limiting. It will be readily understood that the aspects of the present disclosure, as generally described herein and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means +/−5%.

As used herein, “osteostimulative” refers to the ability of a material to improve healing of bone injuries or defects.

As used herein, “osteoconductive” refers to the ability of a material to serve as a scaffold for viable bone growth and healing.

As used herein, “osteoinductive” refers to the capacity of a material to stimulate or induce bone growth.

As used herein, “biocompatible” refers to a material that elicits no significant undesirable response when inserted into a recipient (e.g., a mammalian, including human, recipient).

As used herein, “resorbable” refers to a material's ability to be absorbed in-vivo through bodily processes. The absorbed material may turn into bone in the patient's body.

The present disclosure provides an orthopedic implant suitable for use in orthopedic surgery. The orthopedic implant described herein may be used in conjunction with cement or bone void fillers. Further, the orthopedic implant described herein includes a magnesium phosphate porous coating that produces increased fixation strength.

In particular, the present disclosure provides an orthopedic implant that includes an elongated member having a first end and a second end opposite the first end. The orthopedic implant further includes a porous coating secured to an exterior surface of the elongated member. The porous coating includes magnesium phosphate. Such a porous coating includes interconnecting networks of pores which are similar to those of trabecular bone, and may serve to promote bone ingrowth deeper into the porous coating and hence provide better long-term orthopedic implant fixation. As used herein, “magnesium phosphate” is a general term for salts of magnesium and phosphate appearing in several forms and several hydrates including, but not limited to, monomagnesium phosphate ((Mg(H₂PO₄)₂)·xH₂O), dimagnesium phosphate ((MgHPO₄)·xH₂O), and trimagnesium phosphate ((Mg₃(PO₄)₂)·xH₂O).

In one example, the porous coating further includes KH₂PO₄ in an amount between about 20-70 dry weight percent, MgO in an amount between 10-50 dry weight percent, a calcium containing compound, and a poly-lactic acid. In such an example, the poly-lactic acid comprises one of Poly(L-lactic acid) PLA, poly(L, DL-lactide) PLDLA, and poly(L-lactide-co-glycolide) PLGA. Further, the porous coating may include a bioactive therapeutic agent. Such bioactive therapeutic agents may include natural or synthetic therapeutic agents such as bone morphogenic proteins (BMPs), growth factors, bone marrow aspirate, stem cells, progenitor cells, antibiotics, amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin, or other osteoconductive, osteoinductive, osteogenic, bio-active, or any other fusion enhancing material or beneficial therapeutic agent.

In one example, the porous coating further comprises a sugar, and wherein the sugar comprises one of sugar alcohols, sugar acids, amino sugars, sugar polymers glycosaminoglycans, glycolipds, sugar substitutes and combinations thereof. A thickness of the porous coating on the exterior surface of the elongated member ranges from 200 μm to 50 mm. In some examples, the porous coating does not cover the entirety of the exterior surface of the elongated member such that there are areas of bare titanium polyetheretherketone (PEEK), polyurethane, and/or bone. In another example, the entire exterior surface of the elongated member is covered with the porous coating.

The porous coating may secured to the elongated member of the orthpedic implant in a variety of ways. In one example, the porous coating comprises a powder with a particle size between 10 μm and 200 μm. In such an example, an energy source (e.g., a laser or electron beam) is used with the powder to build structures layer by layer, selectively sintering powder together so as to build a three dimensional shape. More particularly, a thin layer of the powder is spread out as a uniform layer, and then the energy source is used to selectively melt regions of the powder, fusing the particles together. Another layer of powder is then spread on top of the first layer, and the energy source again melts regions of the powder. This process is continued until the complete three-dimensional dynamic porous coating is built. It is possible to manufacture sheets of the porous coating that can then be wrapped around the orthopedic implant, sleeves of the dynamic porous coating that can be slid over the orthopedic implant, or create the porous coating directly onto the orthopedic implant.

In another example, the porous coating is formed of sintered layers of powder that create a three-dimensional porous coating. In particular, multiple thin sheets of material may be laminated one on top of another. A pattern can be chemically etched, punched, or cut out of each of the sheets and, by altering the geometry of the pattern on each sheet, it is possible to create a porous dodecahedron or other multi-facet structure which can function as a porous coating for the orthopedic implant. More particularly, each sheet can be layered one on top of another and sintered together so as to create a porous structure. By changing the geometry of the cut-out on each layer, it is possible to create many different porous structures.

In another example, a structure similar to trabecular bone (e.g., a polyurethane foam) is coated with another material (e.g., a magnesium phosphate material) by vapor deposition, low temperature arc vapor deposition (LTAVD), chemical vapor deposition, ion beam assisted deposition and/or sputtering. The underlying structure (e.g., the polyurethane foam) may then undergo pyrolysis so as to remove the underlying structure (e.g., the polyurethane foam), leaving a magnesium phosphate based metallic structure which can be attached to the orthopedic implant (e.g., by sintering, brazing, diffusion bonding, gluing or cementing, etc.).

In one particular example, commercial pure titanium (ASTM F67, grade 2) sheets may be cut into disks of diameter 16 mm and thickness 1 mm. After cutting, the titanium sheets may be ground with SiC abrasive papers, polished to a mirror surface, rinsed with acetone in an ultrasonic bath, and washed with distilled water. In one particular example, the electrolyte used may contain 0.042 M Ca(NO3)2 and 0.025 MNH4H2PO4 and its pH value may be about 4. Cathodic polarization and deposition may be carried out with EG&G Mode1273A potentiostat/galvanostat. The titanium disks may be used as the cathode, a Pt plate as the counter-electrode, and a silver/silver chloride electrode in saturated potassium chloride (0.197 V vs. SHE) as the reference electrode. Cathodic polarization may be conducted from open circuit voltage to 3.0 V (vs. Ag/AgCl electrode) at a rate of 0.6 V/h. Magnesium coatings may then be deposited at current densities of 1-20 mA/cm2 for a duration of 5-40 minutes at room temperature (25 jC). After deposition, the specimens may be rinsed in distilled water to remove residual electrolyte and dried in air for 24 h. In some examples, specimens deposited at 10 mA/cm2 were annealed at 100-700 jC for 1 hour.

Further, curing the uncured osteostimulative material may comprise heat treating the orthopedic implant after the uncured osteostimulative material is applied to the exterior surface of the orthopedic implant. Because high deposition temperature is needed in order to obtain high quality of MgO films, the curing temperature may be varied from 400° C. to 500° C. in 25° C. intervals. The annealing curing is inversely proportional to the thickness of the mixture of osteostimulative material. After cooking in an oven, the orthopedic implant may then be air dried. The process of heat treating will reduce drying time exponentially compared with just applying the osteostimulative material to the exterior surface of the orthopedic implant and allowing it to cure without the aid of heat.

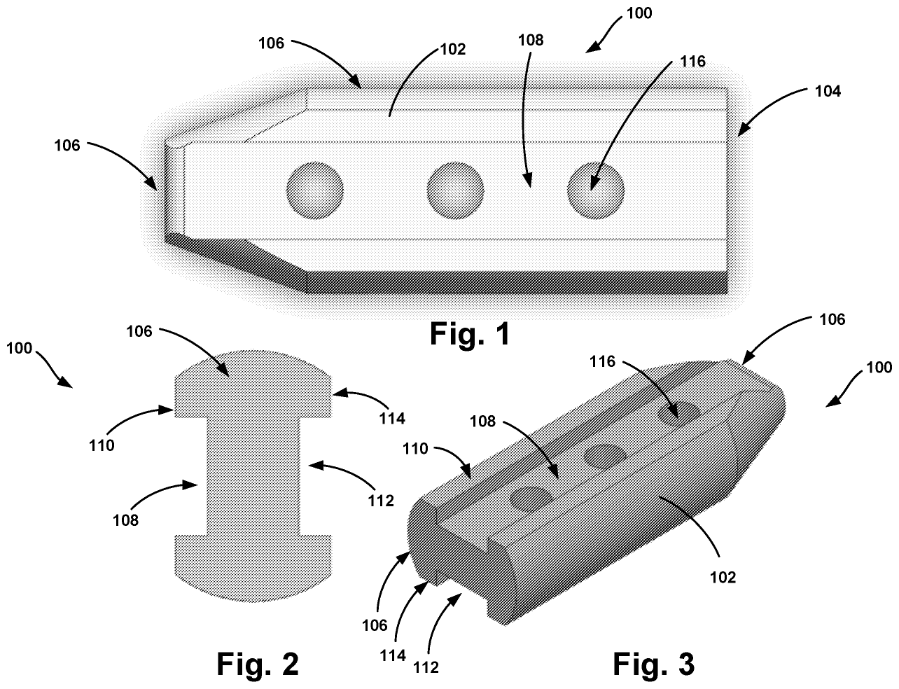

The orthopedic implant make take a variety of forms. In one example, as shown in FIGS. 1-3, orthopedic implant 100 includes an elongated member 102 having a first end 104 and a second end 106 opposite the first end 104. The elongated member 102 is tapered at the second end 106 such that a width of the second end 106 is less than a width of the first end 104.

In one example, the orthopedic implant 100 further includes a first channel 108 positioned on a first side 110 of the elongated member 102 and extending from the first end 104 to the second end 106. In such an example, the orthopedic implant 100 further includes a second channel 112 positioned on a second side 114 of the elongated member 102 and extending from the first end 104 to the second end 106. The orthopedic implant 100 further includes one or more through holes 116 connecting the first channel 108 to the second channel 112. The first channel 108 is recessed in the first side 110 of the elongated member 102, and the second channel 112 is recessed in the second side 114 of the elongated member 102.

Further, the orthopedic implant 100 may include a first portion where the first side 110 and the second side 114 are a fixed distance apart, and a second portion that is tapered. The first side 110 may have a radius of curvature for at least the first portion, and the second side 114 may have the radius of curvature for at least the first portion

In one example, the exterior surface of the elongated member 102 is unthreaded. In such an example, the first end 104 may be substantially flat, such that it is configured to be inserted into a structure via a hammer or other similar tool. In another example, at least a portion of the exterior surface of the elongated member 102 includes a plurality of threads. In such an example, the orthopedic implant 100 may also a drive socket positioned at the first end 104 of the elongated member 102. Such a drive socket may comprise a recessed cutout in the first end 104 of the elongated member 102. In one example, the orthopedic implant 100 also includes a head coupled to the first end 104 of the elongated member 102. The head may have a diameter greater than a diameter of the elongated member 102, thereby providing a stopping point for the orthopedic implant 100 as it is inserted into a structure when in use. The elongated member 102 may be integral to the head such that they are formed unitarily, or the elongated member 102 may be coupled separately to the head. In such an example, the drive socket may be integral to the head, such that the drive socket comprises a recessed cutout in the head.

Such a drive socket is designed to interact with a suitable torque-transmitting insertion device, such as an implant driver, and thereby allow transmission of the requisite amount of torque needed to drive the implant into the prepared socket. For example, the drive socket may be a polygonal recess in the first end of the elongated member while the torque-transmitting feature characterizing the distal end of the driver is a corresponding polygonal protrusion (such as found on the conventional hex key or “Allen” key). In another embodiment, the drive socket may be one or more axially extending slots recessed in the first end of the elongated member 102 while the driver is a slotted, flat blade or crosshead (“Phillips head”) screwdriver. However, other embodiments will be readily apparent to the skilled artisan. Moreover, it will be readily understood by the skilled artisan that the position of the respective coordinating elements (e.g., recessed slots and grooves that mate with assorted projecting protrusions, protuberances, tabs and splines) may be exchanged and/or reversed as needed.

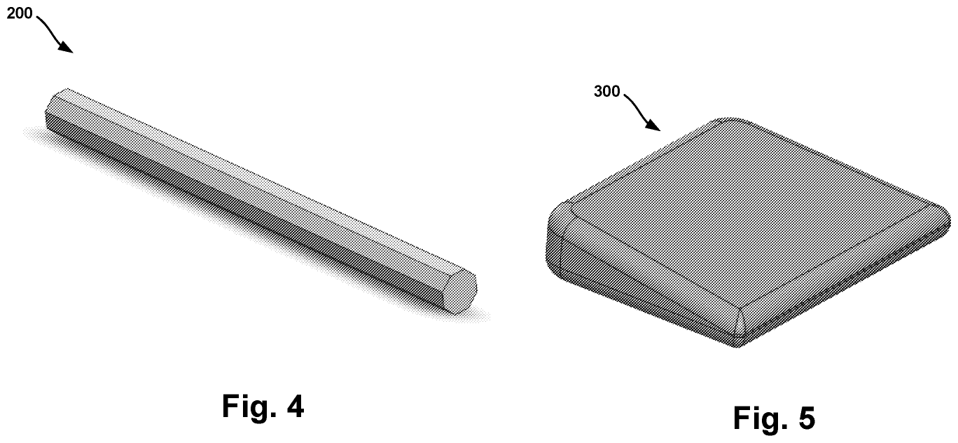

In another example, as shown in FIG. 4, the orthopedic implant comprises a pin 200. In another example, as shown in FIG. 5, the orthopedic implant comprises a wedge 300. Other forms of orthopedic implants are possible as well with the method described above.

The elongated member may take a variety of forms. In one example, the elongated member of the orthopedic implant may comprise titanium, polyetheretherketone (PEEK), polyurethane, bone, or combinations thereof.

In another example, the entirety of the orthopedic implant is made from a cured osteostimulative material including a polymer including poly-lactic acid and either magnesium phosphate or potassium phosphate. As used herein, “poly-lactic acid” or polylactide (PLA) is a biodegradable and bioactive thermoplastic aliphatic polyester derived from renewable resources, and may take a variety of forms including, but not limited to, poly-L-lactide (PLLA), poly-D-lactide (PDLA), and poly(L-lactide-co-D,L-lactide) (PLDLLA). As used herein, “magnesium phosphate” is a general term for salts of magnesium and phosphate appearing in several forms and several hydrates including, but not limited to, monomagnesium phosphate ((Mg(H₂PO₄)₂)·xH₂O), dimagnesium phosphate ((MgHPO₄)·xH₂O), and trimagnesium phosphate ((Mg₃(PO₄)₂)·xH₂O). As used herein, “calcium phosphate” is a family of materials and minerals containing calcium ions (Ca′) together with inorganic phosphate anions and appearing in a variety of forms including, but not limited to, monocalcium phosphate, dicalcium phosphate, tricalcium phosphate, octacalcium phosphate, amorphous calcium phosphate, dicalcium diphosphate, calcium triphosphate, hydroxyapatite, apatite, and tetracalcium phosphate.

Making the entirety of the elongated body of the orthopedic implant from a polymer including poly-lactic acid and either magnesium phosphate or potassium phosphate has a number of advantages. In particular, such a material allows the orthopedic implant to be absorbed in-vivo, producing increased fixation strength and faster absorption into the body. In contrast to traditional metal alloy bone screws, there is no need to remove the orthopedic implant after a certain period of time because the material of the orthopedic implant enables bone to actually replace the structure of the orthopedic implant. As such, there is no void that is left behind after the orthopedic implant is absorbed in-vivo. Instead, the orthopedic implant is replaced with bone structure grown naturally in the body and the resulting fixation strength is very strong. As such, the elongated member of the orthopedic implant may be completely resorbable.

The resultant orthopedic implant exhibits relatively high mechanical strength for load bearing support, while additionally and desirably providing high osteoconductive and osteoinductive properties to achieve enhanced bone ingrowth and fusion. In use, the polymer including poly-lactic acid and either magnesium phosphate or potassium phosphate that makes up the orthopedic implant will induce bone growth into the orthopedic implant and be resorbed. The orthopedic implant is eventually replaced by bone in the body, thereby firmly securing the component to which the orthopedic implant (e.g., a substitute ligament in an ACL reconstruction surgery or an existing rotator cuff in a rotator cuff reattachment surgery) is connected to the bone structure the body.

In yet another example, at least a portion of an exterior surface of the elongated member includes a cured osteostimulative material onto which the porous coating is positioned. Such an osteostimulative material may take a variety of forms. The osteostimulative material may allow for in-situ (i.e., in vivo) attachment of biological structures to each other and to manmade structures. The osteostimulative material may also facilitate the repair of bone, ligaments, tendons and adjacent structures. The osteostimulative material may also provide a bone substitute for surgical repair. The formulation of the osteostimulative material is usable at numerous temperatures, pH ranges, humidity levels, and pressures. However, the formulation can be designed to be utilized at all physiological temperatures, pH ranges, and fluid concentrations. The osteostimulative material typically is, but not necessarily, injectable before curing and can exhibit neutral pH after setting. It may be absorbed by the host over a period of time.

In one particular example, the cured osteostimulative material comprises KH₂PO₄ in an amount between about 20-70 dry weight percent, Magnesium oxide (MgO) in an amount between 10-50 dry weight percent, a calcium containing compound, a poly-lactic acid, and either magnesium phosphate or potassium phosphate. The cured osteostimulative material may have both osteoconductive and osteoinductive properties. In addition, the cured osteostimulative material may be bioresorbable. A thickness of the cured osteostimulative material on the exterior surface of the elongated member may range from about 200 μm to about 50 mm. In some examples, the cured osteostimulative material does not cover the entirety of the exterior surface of the elongated member such that there are areas of bare titanium polyetheretherketone (PEEK), polyurethane, and/or bone. In another example, as discussed above, the exterior surface of the elongated member may include a plurality of threads or a plurality of grooves. In such an example, the cured osteostimulative material is positioned in one or more of the plurality of grooves. In another example, the entirety of the elongated member comprises the cured osteostimualtive material.

In accordance with a further aspect of the invention, the orthopedic implant may additionally carry one or more bioactive therapeutic agents for achieving further enhanced bone fusion and ingrowth. Such bioactive therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, bio-active, or any other fusion enhancing material or beneficial therapeutic agent. In another example, the bioactive therapeutic agent comprises one of amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin.

The resultant orthopedic implant exhibits relatively high mechanical strength for load bearing support, while additionally and desirably providing high osteoconductive and osteoinductive properties to achieve enhanced bone ingrowth and fusion. In use, the cured osteostimulative material positioned on the exterior surface of the elongated member of the orthopedic implant will induce bone growth into the orthopedic implant and be resorbed. The osteostimulative material is eventually replaced by bone, thereby more firmly embedding the orthopedic implant in the body.

The osteostimulative material is particularly useful in situations (such as plastic surgery) when the use of metallic fasteners and other non-bioabsorbable materials are to be assiduously avoided. The osteostimulative material also is useful when a certain amount of expansion or swelling is to be expected after surgery, e.g., in skull surgeries. It is a good platform for bone-formation. The osteostimulative material can be also used as an anchoring device or grafting material.

In operation, the present invention provides a method for securing an orthopedic implant to a bone, the method comprising: (a) providing the orthopedic implant of any of the embodiments described above, (b) forming a cavity in the bone, and (c) inserting the second end of the elongated member of the orthopedic implant into the cavity in the bone.

In one embodiment, the method further includes applying an uncured osteostimulative material to an exterior surface of the elongated member.

In one embodiment, the method further includes inserting a ligament in the cavity in the bone, and inserting the second end of the elongated member of the orthopedic implant into the cavity in the bone such that the elongated member fills a substantial portion of the cavity, wherein the ligament is securely fixed between the elongated member and an inner surface of the cavity in the bone.

In some examples, one or more components of the orthopedic implant described above is made via an additive manufacturing process using an additive-manufacturing machine, such as stereolithography, multi jet modeling, inkjet printing, selective laser sintering/melting, and fused filament fabrication, among other possibilities. Additive manufacturing enables one or more components of the orthopedic implant and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the orthopedic implant using a Computer Aided Design (CAD) of the orthopedic implant as instructions. As a result, changes to the design of the orthopedic implant can be immediately carried out in subsequent physical creations of the orthopedic implant. This enables the components of the orthopedic implant to be easily adjusted or scaled to fit different types of applications (e.g., for use in various wing sizes). In one particular example, the step of securing the porous coating to the exterior surface of the elongated member of the orthopedic implant comprises performing an additive-manufacturing process to deposit the porous coating on the exterior surface of the elongated member.

The layer-upon-layer process utilized in additive manufacturing can deposit one or more components of the orthopedic implant with complex designs that might not be possible for devices assembled with traditional manufacturing. In turn, the design of the orthopedic implant can include aspects that aim to improve overall operation. For example, the design can incorporate physical elements that help redirect stresses in a desired manner that traditionally manufactured devices might not be able to replicate.

Additive manufacturing also enables depositing one or more components of the orthopedic implant in a variety of materials using a multi-material additive-manufacturing process. In such an example, the elongated member may be made from a first material and the porous coating may be made from a second material that is different than the first material. In another example, both the elongated member and the porous coating are made from the same material. Other example material combinations are possible as well. Further, one or more components of the orthopedic implant can have some layers that are created using a first type of material and other layers that are created using a second type of material. In addition, various processes are used in other examples to produce one or more components of the orthopedic implant. These processes are included in table 1.

TABLE 1

| | |
|---|---|
| DEP | Direct Energy Deposition |
| DMLS | Direct Metal Laser Sintering |
| DMP | Direct Metal Printing |
| EBAM | Electron Beam Additive Manufacturing |
| EBM | Electron Beam Leting |
| EBPD | Electron Beam Powder Bed |
| FDM | Fused Deposition Modeling |
| IPD | Indirect Power Bed |
| LCT | Laser Cladding Technology |
| LDT | Laser Deposition Technology |
| LDW | Laser Deposition Welding |
| LDWM | Laser Deposition Welding with integrated Milling |
| LENS | Laser Engineering Net Shape |
| LFMT | Laser Freeform Manufacturing Technology |
| LMD-p | Laser Metal Deposition-powder |
| LMD-w | Laser Metal Deposition-wire |
| LPB | Laser Powder Bed |
| LPD | Laser Puddle Deposition |
| LRT | Laser Repair Technology |
| PDED | Powder Directed Energy Deposition |
| SLA | Stereolithography |
| SLM | Selective Laser Melting |
| SLS | Selective Laser Sintering |
| SPD | Small Puddle Deposition |

Each of the components of the orthopedic implant described above may represent a module, a segment, or a portion of program code, which includes one or more instructions executable by a processor or computing device for creating such devices using an additive-manufacturing system. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Because many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. An orthopedic implant, comprising:
an elongated member having a first end and a second end opposite the first end; and
a porous coating secured to an exterior surface of the elongated member, wherein the porous coating includes magnesium phosphate.

2. The orthopedic implant of claim 1, wherein the porous coating further includes KH₂PO₄ in an amount between about 20-70 dry weight percent, MgO in an amount between 10-50 dry weight percent, a calcium containing compound, and a poly-lactic acid.

3. The orthopedic implant of claim 2, wherein the poly-lactic acid comprises one of Poly(L-lactic acid) PLA, poly(L, DL-lactide) PLDLA, and poly(L-lactide-co-glycolide) PLGA.

4. The orthopedic implant of claim 2, wherein the porous coating further includes a bioactive therapeutic agent.

5. The orthopedic implant of claim 4, wherein the bioactive therapeutic agent comprises one of amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin.

6. The orthopedic implant of claim 2, wherein the porous coating further comprises a sugar, and wherein the sugar comprises one of sugar alcohols, sugar acids, amino sugars, sugar polymers glycosaminoglycans, glycolipds, sugar substitutes and combinations thereof.

7. The orthopedic implant of claim 1, wherein a thickness of the porous coating on the exterior surface of the elongated member ranges from 200 μm to 50 mm.

8. The orthopedic implant of claim 1, wherein the elongated member comprises titanium, polyetheretherketone (PEEK), polyurethane, bone, or a cured osteostimulative material.

9. The orthopedic implant of claim 1, wherein the elongated member is tapered at the second end such that a width of the second end is less than a width of the first end.

10. The orthopedic implant of claim 9, further comprising:
a first channel positioned on a first side of the elongated member and extending from the first end to the second end;
a second channel positioned on a second side of the elongated member and extending from the first end to the second end; and
one or more through holes connecting the first channel to the second channel.

11. The orthopedic implant of claim 10,
wherein the one or more through holes comprises a plurality of through holes.

12. The orthopedic implant of claim 10, wherein the first channel is recessed in the first side of the elongated member, and wherein the second channel is recessed in the second side of the elongated member.

13. The orthopedic implant of claim 1, wherein the porous coating comprises a powder with a particle size between 10 μm and 200 μm.

14. The orthopedic implant of claim 1, wherein the porous coating is formed of sintered layers of powder that create a three-dimensional porous coating.

15. The orthopedic implant of claim 1, wherein the porous coating is formed via a chemical vapor deposition process.

16. A method for securing an orthopedic implant to a bone, the method comprising:
providing the orthopedic implant of claim 1;
removing a portion of the bone to create a cavity; and
inserting the second end of the elongated member of the orthopedic implant into the cavity in the bone.

17. The method of claim 16, further comprising:
applying an uncured osteostimulative material to an exterior surface of the elongated member.

18. The method of claim 16, further comprising:
inserting a ligament in the cavity in the bone; and
inserting the second end of the elongated member of the orthopedic implant into the cavity in the bone such that the elongated member fills a substantial portion of the cavity, wherein the ligament is securely fixed between the elongated member and an inner surface of the cavity in the bone.
